(19) United States
(12) Patent Application Publication
(10) Pub. No.: US 2009/0054795 A1
(43) Pub. Date: Feb. 26, 2009

(54) METHOD FOR GENERATING THREE STANDARD SURFACE ECG LEADS DERIVED FROM THREE ELECTRODES CONTAINED IN THE MID-HORIZONTAL PLANE OF THE TORSO

(76) Inventors: Dale J. Misczynski, Austin, TX (US); Vladislav Bukhman, East Northport, NY (US); Mykola Budnyk, Kyiv (UA); Illya Chaykovsky, Kyiv (UA)

Correspondence Address: WINSTEAD PC, P.O. BOX 50784, DALLAS, TX, 75201, US

(21) Appl. No.: 11/843,183

(22) Filed: Aug. 22, 2007

(51) Int. Cl. A61B 5/0452

(52) U.S. Cl. 600/509

(57) ABSTRACT

A method for a 3-lead electrocardiographic (ECG) recording comprising three signal electrodes contained in the mid-horizontal plane of the human torso and the calculation of the standard leads I, II and III. Such electrodes are placed in-line as in a chest belt instead of the traditional positioning of electrodes in the upper and low parts of the frontal plane of the torso.

### TECHNICAL FIELD

The present invention relates to the medical diagnostic techniques intended for measurements of electric signals originated by human or animal heart. More particularly, the principles of the present invention are devoted to cardiology and are targeted to revealing pathological peculiarities of electrophysiological processes into the myocardium. In detail, the present invention relates to a design of a device for electrocardiography (ECG) recording, and method, which provides the source for the visualization of signals in view of II, III standard ECG leads, and I.

### BACKGROUND INFORMATION

Vital activity of a living organism is accompanied by generation of electric potentials, which give a source for bioelectric measurements, among them the ECG is most widespread, known and clinically significant.

ECG has a number of advantages as compared with measurements of other physical quantities such as ultrasound, MRI, coronary angiography, radionuclide scintigraphy, invasive electrophysiology tests, magnetocardiography, biochemical analyses, etc. The main ECG advantages are as follows: non-invasive recording; safety and harmlessness; relative simplicity of using; non-expensive apparatus; pictorial information and quick interpretation; possibility for portable and wearable devices; possibility for long-time monitoring of patients status aimed to monitoring of pharmacological treatment or surgical invasions.

However, the essential drawback of the ECG method is poor sensitivity and specificity. For example, according to Connolly [Connolly D C., Elveback L R., Oxman H A. Coronary heart disease in residents of Rochester, Minn.: Prognostic value of resting electrocardiogram at the time of initial diagnosis of angina pectoris. Mayo Clin. Proc. 1984; 59:247-50] the standard 10-second ECG at rest is “normal” nearly at 50% of patients with chronic coronary artery disease (CAD). In order to increase the diagnostic yield of the ECG, clinicians use ambulatory monitoring. The problem with the ambulatory monitoring is in the placement of electrodes which should be done by trained professionals. If electrodes are misplaced or fall apart during recording, the ECG may lose its validity or became worthless unless the electrodes are fixed by the trained personal.

The present invention provides a method and simple and easy-to-use apparatus for ECG recording which does not require assistance of medical professionals while providing the standard 3-leads ECG recording.

FIG. 2A is a spatial presentation of the positioning of electrodes for the standard 3-lead ECG. R means the right shoulder, L—left shoulder, and F—low abdomen. In current electrocardiography, it is assumed that the triangle formed by R, L and F is equilateral. The applied rectangular coordinate system is formed by the X-axis directed to the left, Y-axis directed to the head, and the Z-axis directed to the back of the human torso. XOY is the frontal plane of the torso and XOZ is mid-horizontal plane of the torso. E is the resultant electric heart vector (EHV). Ex and Ey are components of EHV registered within standard ECG lead system. The new electrodes placement system is created by rotating the coordinate system by 90° around axis OX (FIG. 2B). EHV components registered by such lead system are Emx and Emz (FIG. 2B). As the result, all three electrodes are in mid-horizontal plane instead of the frontal plane of the traditional 3-lead system. The mid-horizontal positioning of the electrodes generates modified leads, mI, mII and mIII where

mI=mL−mR, mII=mF−mR, mIII=mF−mL (1)

Where:

mL, mR, and mF—potentials measured at Left, Right and Back electrode positions (FIG. 2B).

Assuming that the triangles formed by the electrodes are equilateral, the electric signals generated by the human heart are described by Equations (2-3)

I=Ex, II=(Ex+√3Ey)/2; III=II−I=(−Ex+√3Ey)/2 (2)

mI=Emx, mII=(Emx+√3Emz)/2; mIII=(−Emx+√3Emz)/2 (3)

Where:

Ex, Ey, Ez are amplitudes of EHV components registered by standard frontal plane leads system.

Emx, Emy, Emz are amplitudes of EHV components registered by horizontal plane leads system. By comparing Equations (2) and (3) we can see that leads I and ml register the EHV projections onto the same direction, i.e. axis OX and their amplitudes are only differed by some scaling coefficient k1.

Based on the above assumption, the ratio between projections Ex and Emx is constant and is not time dependant because I and mI leads did not depended on the EHV direction. Contrary, pairs (II, mII) and (III, mIII) vary upon the changing of EHV directions resultant of different angles formed by the vector E and the frontal plane and the vector E and the horizontal plane.

The scaling coefficient k1 is calculated by equation (4).

k1=Ex/Emx=I/mI (4)

Furthermore, the coefficient k1 is only determined by EHV amplitudes, which are registered by different leads system, and represent the ratio of vectors E and Em (5), where Em is amplitude of EHV registered within lead system formed by mid-horizontal electrode placement.

k1=E/Em (5)

It is known that the ratio of two vectors is equal to ratios of their projections (6).

k1=Ey/Emy=Ez/Emz (6)

Therefore, the correlations between projections of the standard frontal electrode placement and the mid-horizontal electrode placement are determined by Equations (7).

Ex=k1*Emx; Ey=k1*Emy; Ez=k1*Emz (7)

Furthermore, Equations (8) are received by combining the Equations (3) and Equations (7).

I=k1*Emx; II=k1*(Emx+√3Emy)/2; III=k1*(−Emx+√3Emy)/2 (8)

Finally, considering that lead mI and X-projection of EHV, Emx are equal (see Equation (3)) we obtain the Equations (9) which determine values of the standard leads I, II and III using mid-horizontal electrodes placement.

I=k1*mI; II=k1*(√3Emy+mI)/2; III=k1*(√3Emy−mI)/2 (9)

The solution of Equations (9) represents an inverse, ill-posed problem, because unknown EHV component Emy cannot be measured by the frontal standard electrode placement. The inverse problem consists in using the results of actual observations to infer the values of the parameters characterizing the system under investigation. The correct solution of the above-mentioned inverse problem of the recalculation is impossible with utilizing the single coefficient k1.

Three coefficients should be introduced instead of a single one in order to solve above problem. In the present invention, the values of coefficients for the conversion of the modified leads, mI, mII and mIII to standard leads I, II and III are obtained from the observed data received from the standard, frontal lead placement and horizontal lead placement by Equation (10).

k1=I/mI, k2=II/mII and k3=III/mIII (10)

Coefficients k2 and k3 vary during cardiac cycle due to changes in the direction of EHV (6) and changes of angles between EHV and the frontal and horizontal planes.

The measurements of the signal from horizontal plane placement of electrodes are calculated by Equation (11).

mI=k1*mL−k1*mR, mII=k2*mF−k2*mR, mIII=k3*mF−k3*mL (11)

First coefficient k1 is constant because the non-dipole contribution is negligible. Therefore, the deviation of k1 during cardiac cycle may serve as the criterion of the accuracy of the calculation of k2 and k3 within the framework of embodiment.

### SUMMARY

One aspect of the present invention is the obtaining of the ECG signal from the electrodes placed in the mid-horizontal plane of the human torso and converting the signal values onto a standard 3-lead ECG system.

The proposed present invention provides a new way of obtaining the standard ECG by placing electrodes into a belt-type holder.

In accordance with the principles of the present invention, the belt is mounted at the human thorax and includes at least three signal electrodes mR, mL, and mF. mR and mL are placed in the 5^{th} Intercostal Space at the Left and Right Anterior Axillary Lines. mF is placed in the 5^{th} Intercostal Space at the Posterior Axillary Line (FIG. 1).

### BRIEF DESCRIPTION OF THE DRAWINGS

A better understanding of the present invention can be obtained when the following detailed description is considered in conjunction with the following drawings, in which:

FIG. 1 illustrates the arrangement of electrodes on a proposed device (Cardio-belt) placed onto a human torso in accordance with an embodiment of the present invention;

FIG. 2 illustrates the mutual relation between conventional ECG leads, I, II and III and the proposed mid-frontal plane lead placement in accordance with an embodiment of the present invention;

FIG. 3 illustrates waveforms of the ECG signals during the cardiocycle at I, II, III standard leads and mI, mII, mIII mid-frontal leads recorded simultaneously for a volunteer C from the learning group in accordance with an embodiment of the present invention;

FIG. 4A illustrates waveforms (solid line represents the Standard Lead I, dot line represents mid-frontal lead mI, and dash line represents the sum of mid-frontal leads mI and mII (mI+mII)) of the ECG signals acquired from the volunteer C from the learning group in accordance with an embodiment of the present invention;

FIG. 4B illustrates the plot of normalized coefficient k1N, which is the ratio between the standard lead I and sum of mid-frontal leads mI and mII, acquired from the volunteer C from the learning group in accordance with an embodiment of the present invention;

FIG. 5A illustrates the plot of normalized coefficient K1N calculated over a learning group in accordance with an embodiment of the present invention;

FIG. 5B illustrates ECG waveforms (solid asterisked line represents the recorded signal at the Standard lead I and the dash line shows the calculated Standard lead I based on the predefined coefficient K1N (FIG. 5A) and the sum mI and mII) of the recorded and the calculated Standard lead I acquired from the control person N not including into learning group in accordance with an embodiment of the present invention;

FIGS. 6A and 6B illustrate plots of the coefficients K2N (A) and K3N (B) during the cardiocycle, which are calculated based on averaging of ECG data of the learning group in accordance with an embodiment of the present invention; and

FIGS. 7A and 7B illustrate ECG waveforms of the recorded (solid asterisked line) and the calculated (dash line) Standard Leads II (A) and III (B) acquired from the control person N not including the learning group in accordance with an embodiment of the present invention.

### DETAILED DESCRIPTION

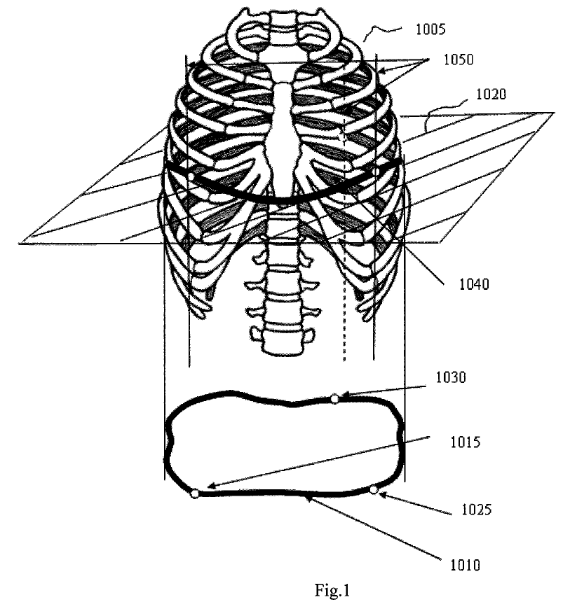

The preferred embodiment of the present invention is illustrated in FIG. 1. A harness with electrodes 1010 is placed on the thorax 1005 in mid-horizontal plane 1020 and includes at least 3 signal electrodes mR 1015, mL 1025, and mF 1030 mounted in such way that mR and mL are positioned in 5^{th} Intercostal Space 1040 at Left and Right Anterior Axillary Lines 1050 while mF is positioned in 5^{th} Intercostal Space 1040 at Posterior Axillary Line 1050.

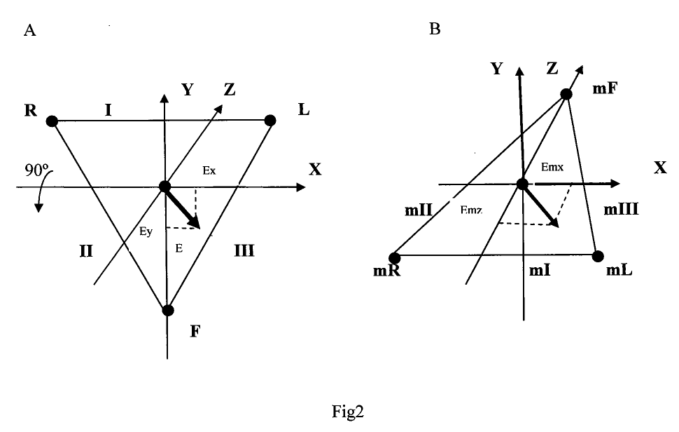

FIG. 2 illustrates the spatial relationship between the Standard 3-lead electrode placement in the frontal plane A and the presented mid-horizontal placement B. The placement B is formed by rotating the triangle RLF forward by 90°. The coordinate projections of the resultant electrical force vector E can be recalculated to the correspondent projections of the Standard placement A. However, it provides an approximate solution due to many interfering factors and the controversy of the generally assumed heart electrical model.

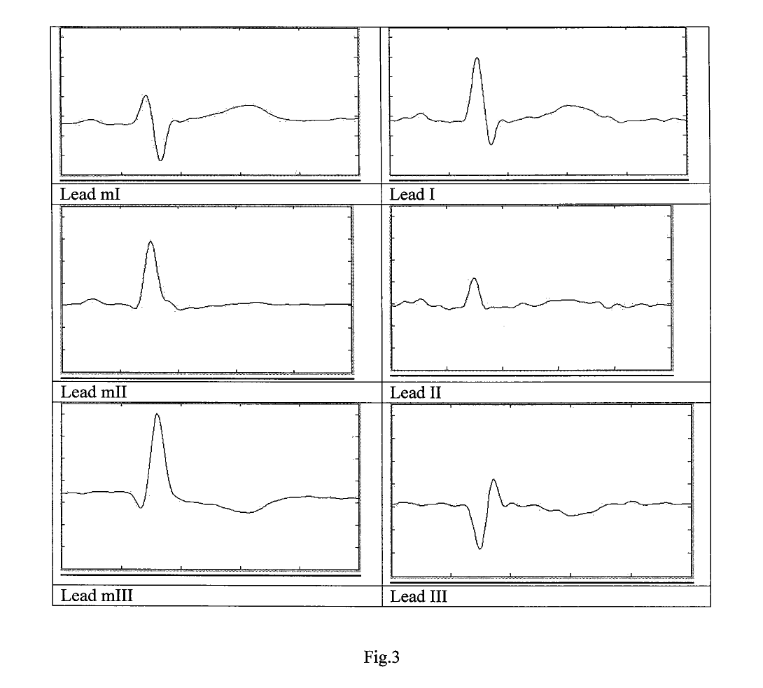

Another feature of an embodiment of the present invention is obtaining the scaling coefficients of the transformation of signal values derived from the mid-horizontal lead placement to the Standard lead placement by using the learning procedure. For this purpose, the signals are acquired simultaneously from the Standard lead placement and the mid-horizontal placement. The scaling coefficients for the cardiac cycle are averaged based on the signal values received from the representative number of volunteers. ECG signals during the cardiocycle at three standard (I, II, III) leads and three mid-frontal plane (mI, mII, mIII) leads for the healthy volunteer are presented at FIG. 3.

The array of 6 sets of ECG strip I(t), II(t), III(t), mI(t), mII(t), mIII(t) are recorded synchronously from the single volunteer. ECG signals are pre-processed (e.g., filtered if needed), and all coefficients (k1j, k2j, k3j) for each from j volunteers are calculated. All coefficients are averaged during cardio-cycles by Equation (12) as using sign denoting time-averaging procedure < . . . >.

k1j(t)=<Ij(t)/mIj(t)>, k2j(t)=<IIj(t)/mIIj(t)>, k3j(t)=<IIIj(t)/mIIIj(t)> (12)

In the next step, above coefficients are averaged onto learning group according to Equations (13)

K1(t)=<<k1j(t)>>, K2(t)=<<k2j(t)>>, K3(t)=<<k3j(t)>> (13)

Where << . . . >> sign denoting averaging procedure onto the learning group.

A feature of the proposed approach consists in that in order to avoid the “dividing-by-zero” problem, maximum and minimum values of each from 6 input signals are pre-determined and normalized signals at all leads are calculated according to (14).

Hereafter the method is illustrated for lead I, because expressions for other leads are analogical.

ΔIj=max{mIj}−min{mIj}, mINj=mIj+ΔIj, INj=Ij+ΔIj (14)

Where:

ΔIj is the peak-to peak deviation of signal mI for j-th volunteer;

mINj is the normalized signal for the horizontal placement for j-th volunteer; and

INj is the normalized signal for the Standard placement for j-th volunteer.

In the final step of the learning procedure, the normalized coefficient for j-th volunteer k1Nj, time-averaged during cardiocycle, and K1N, averaged onto the learning group, are calculated by Equation (15).

k1Nj(t)=<INj/mINj>, K1N(t)=<<k1Nj(t)>> (15)

The standard lead I for any person outside of the learning group, Ii is calculated by the Equation (16).

Ii=K1N*mINi−ΔIj (16)

Where:

ΔIi is the peak-to peak deviation of signal mI for i-th person from control group (not including into the learning group);

mINi is the normalized signal for the proposed placement for above i-th person.

In the next step, the calculated lead I waveforms are compared with the averaged waveforms of the standard lead I which are stored as a Lead I templates. If two signals differ by a predefined threshold, then the calculated signal is included in the learning group and coefficients are recalculated using (15) and stored in the memory by replacing the old template.

In a summary, the presented method provides a calculation of the standard leads I, II and III using modified leads mI, mII and mIII by Equation (16) where K1N (t), K2N (t) and K3N (t) are normalized time-dependant coefficients defined by Equation (15).

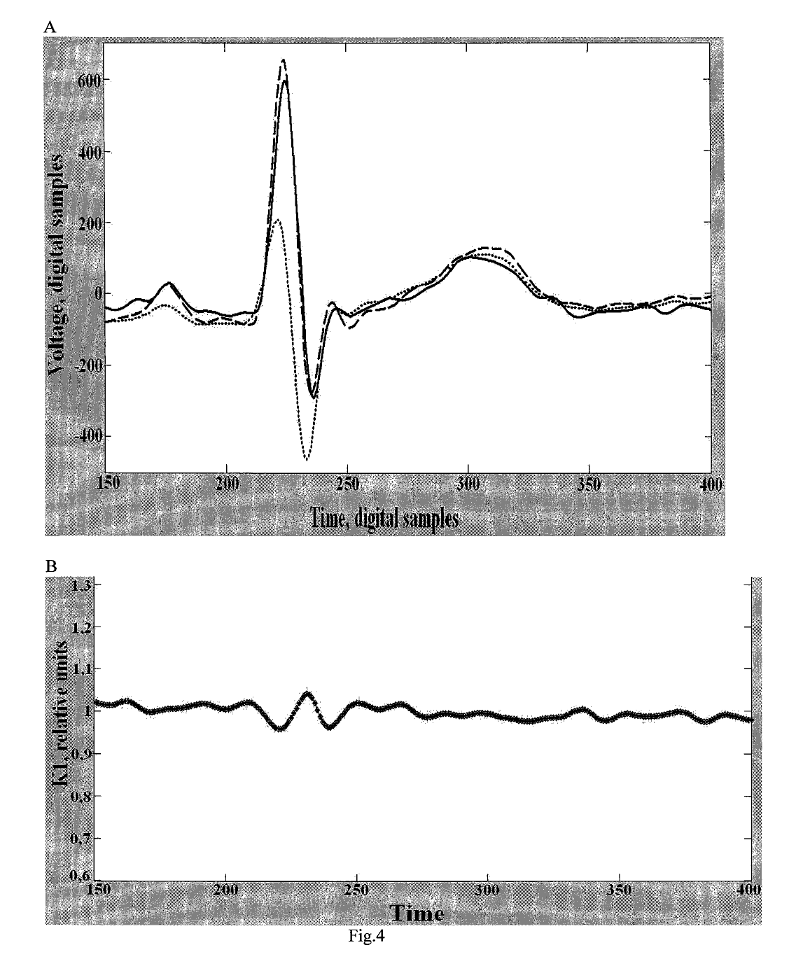

FIG. 4A shows the signal representing lead I (solid line) and lead mI (dot line) acquired from a volunteer C from the learning group. One can see that I differ from mI in spite of the fact that they are both in the same plane. The reason is in the simplification of the above embodiment.

In a more precise approach, the combination of modified leads is utilized. The dash line in FIG. 4A represents the sum of leads mI and mII for the same volunteer. The line almost coincides with lead I. In this approach, the normalized coefficient k1N for j-th volunteer is calculated by Equation (17) instead of the first Equation (15)

k1Nj(t)=<INj(t)/[mINj(t)+mIINj(t)]> (17)

Where:

mIINj is the normalized signal for the proposed placement for j-th volunteer.

The curve shown in FIG. 4B represents the plot of k1Nj(t) during the cardiac cycle for the same volunteer C. The coefficient k1N has a mean value=0.9985 and low variability equal ±8%. It is sufficient to provide reliable and valid waveforms of the standard Lead I.

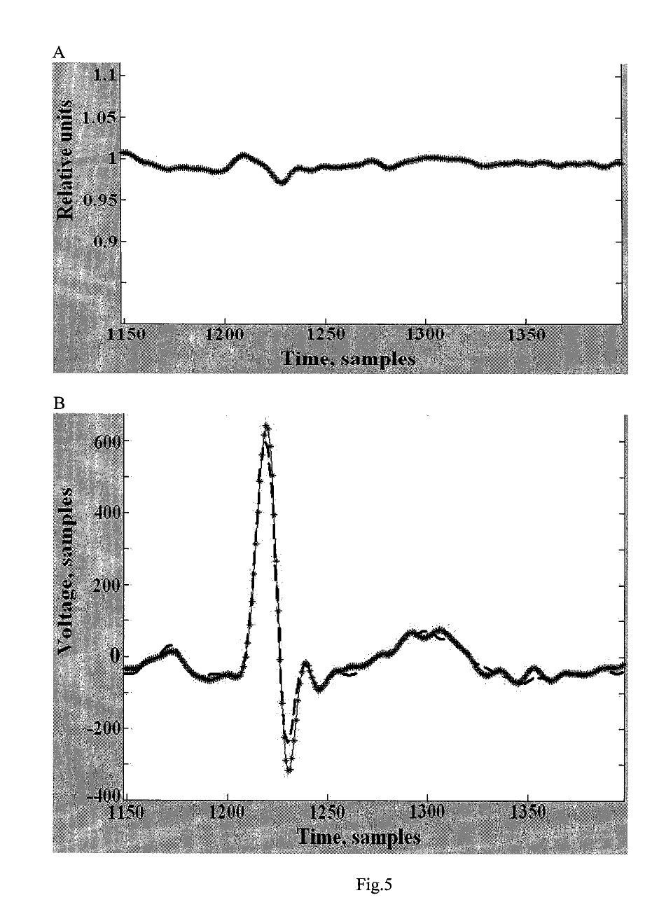

FIG. 5A shows the plot of the coefficient K1N received from the learning group according to (17) and second Equation (15). In this example, K1N equal 0.9985±5%. It may be used for the calculation of the Lead I by the Equation (18) for a patient i outside the learning group.

Ii(t)=K1N(t)*[mINi(t)+mIINi(t)]−(ΔIi+ΔIIi) (18)

Where:

ΔIIi is the peak-to peak deviation of signal mII for i-th person outside from the learning group.

FIG. 5B demonstrates the real Lead I (solid asterisked line) and the calculated Lead I (dash line) acquired from person N outside the learning group.

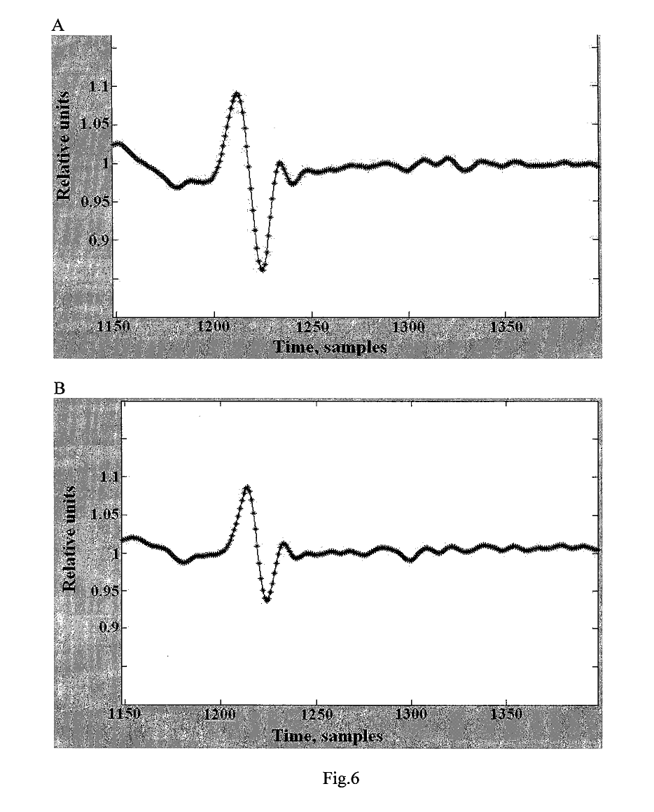

FIG. 6A shows the normalized coefficient K2N averaged for the learning group according to Equation (19).

k2Nj(t)=<IINj/mIINj>, K2N(t)=<<k2Nj(t)>> (19)

Where:

IINj is the normalized signal for the Standard lead II for j-th volunteer.

From FIG. 6A, it is clearly seen that the curve K2N has a QRS type of wave in the area of the QRS complex and small fluctuations outside the QRS.

FIG. 6B illustrates the normalized coefficient K3N calculated by Equations (20) and (21).

k3Nj(t)=<IIINj(t)/[mIIINj(t)−mIINj(t)]>, K3N(t)=<<k3Nj(t)>> (20)

IIi(t)=K3N(t)*[mIIINi(t)−mIINi(t)]−(ΔIIIi−ΔIIi) (21)

Where:

IIINj is the normalized signal for the Standard lead III for j-th volunteer;

mINj is the normalized signal for the horizontal lead mIII for j-th volunteer;

ΔIIIi is the peak-to peak deviation of signal mIII for i-th person outside from the learning group.

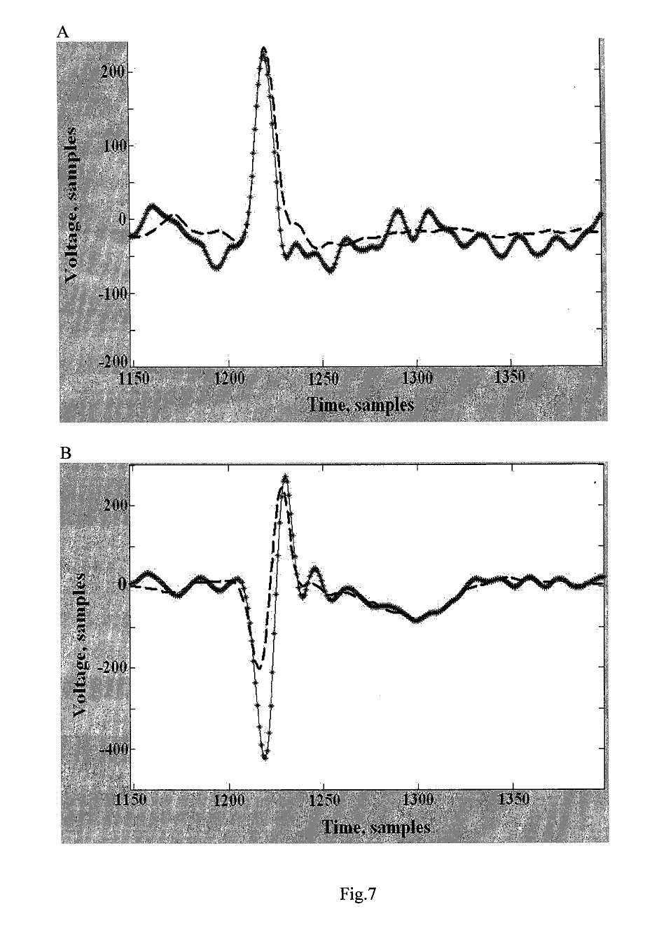

Calculated leads II and III (solid asterisked line) recorded from volunteer N from outside the learning group and standard Lead II and III (dash line) are shown in FIG. 7A and FIG. 7B, respectively.

What is claimed is:

1. A method for obtaining calculated standard leads I, II, and III from placement of electrodes in a mid-frontal plane of a human torso comprising the steps of:
a) positioning at least three signal electrodes in the mid-frontal plane of the human torso;
b) calculating scaling coefficients between standard and modified electrode placement calculated by simultaneous recording of both placements;
c) calculating standard leads from modified leads using the scaling coefficients;
d) adjusting new coefficients if waveforms of calculated lead differ from templates;
e) calculating scaling coefficient for lead I utilizing a sum of lead mI and mII and obtaining a standard lead I from the modified leads;
f) calculating scaling coefficients between standard lead II and modified lead mII; and
g) refining a scaling coefficient for lead III using a difference of lead mIII and lead mII.

2. The method of claim 1, wherein a mid-horizontal placement is formed by rotating a frontal triangle of a standard electrode placement forward by 90°.

3. The method of claim 2, wherein a harness with electrodes is placed on a thorax in a mid-horizontal plane and includes at least three signal electrodes mounted in such way that two of the three signal electrodes are positioned in 5^{th} Intercostal Space at Left and Right Anterior Axillary Lines and a third of the three signal electrodes is positioned in 5^{th} Intercostal Space at Posterior Axillary Line.

4. The method of claim 1, wherein a set of ECGs is obtained from a group of volunteers by simultaneous recording of signals using a standard lead I, II and III placement and mid-horizontal mI, mII and mIII lead placement.

5. The method of claim 4, wherein scaling coefficients during a cardiocycle are calculated for the group of volunteers by (sign < . . . > denoting time-averaging procedure):
k1j(t)=<Ij(t)/mIj(t)>, k2j(t)=<Iij(t)/mIIj(t)>, k3j(t)=<IIIj(t)/mIIIj(t)>.

6. The method of claim 5, wherein the scaling coefficients are averaged into a learning group by (sign << . . . >> denoting averaging procedure over group):
K1(t)=<<k1j(t)>>, K2(t)=<<k2j(t)>>, K3(t)=<<k3j(t)>>.

7. The method of claim 6, wherein a normalized signal is calculated for lead I by:
ΔIj=max{mIj}−min{mIj}, mINj=mIj+ΔIj, INj=Ij+ΔIj;
wherein:
ΔIj is a peak-to peak deviation of signal mI for j-th volunteer;
mINj is a normalized signal for a horizontal placement for the j-th volunteer; and
INj is a normalized signal for a Standard placement for the j-th volunteer.

8. The method of claim 6, wherein a normalized signal is calculated for lead II by:
ΔIIj=max{mIIj}−min{mIIj}, mIINj=mIIj+ΔIIj, IINj=IIj+ΔIIj;
wherein:
ΔIIj is a peak-to peak deviation of signal mII for j-th volunteer;
mIINj is a normalized signal for a horizontal placement for the j-th volunteer; and
IINj is a normalized signal for a Standard placement for the j-th volunteer.

9. The method of claim 6, wherein a normalized signal is calculated for lead III by:
ΔIIIj=max{mIIIj}−min{mIIIj}, mIIINj=mIIIj+ΔIIIj, IIINj=IIIj+ΔIIIj;
wherein:
ΔIIIj is a peak-to peak deviation of signal mIII for j-th volunteer;
mIIINj is a normalized signal for a horizontal placement for the j-th volunteer; and
IIINj is a normalized signal for a Standard placement for the j-th volunteer.

10. The method of claim 7, wherein normalized coefficients for the lead I are time averaged during the cardiocycle by:
k1Nj(t)=<INj/mINj>, K1N(t)=<<k1Nj(t)>>.

11. The method of claim 8, wherein normalized coefficients for the lead II are time averaged during the cardiocycle by:
k2Nj(t)=<IINj/mIINj>, K2N(t)=<<k2Nj(t)>>.

12. The method of claim 9, wherein normalized coefficients for the lead III are time averaged during the cardiocycle by:
k3Nj(t)=<IIINj/mIIINj>, K3N(t)=<<k3Nj(t)>>.

13. The method of claim 1, wherein a standard lead I is calculated by:
Ii=KIN*mINi−ΔIi;
wherein:
ΔIi is a peak-to peak deviation of signal mI for i-th person from a control group; and
mINi is a normalized signal for a proposed placement for above i-th person.

14. The method of claim 1, wherein a standard lead II is calculated by:
Ii=KIIN*mIINi−ΔIi;
wherein:
ΔIIi is a peak-to peak deviation of signal mII for i-th person from a control group; and
mIINi is a normalized signal for a proposed placement for above i-th person.

15. The method of claim 1, wherein a standard lead III is calculated by:
IIIi=KIIIN*mIIINi−ΔIIi;
wherein:
ΔIIIi is a peak-to peak deviation of signal mI for i-th person from a control group; and
mIIINi is a normalized signal for a proposed placement for above i-th person.

16. The method of claim 1, wherein calculated lead I, II and III waveforms are compared with averaged waveforms of standard leads, wherein if two signals differ by a predefined threshold then a calculated signal is included in a learning group and coefficients are recalculated and stored in a memory by replacing an old template.

17. The method of claim 1, wherein normalized coefficients of lead I are calculated by:
k1Nj(t)=<INj(t)/[mINj(t)+mIINj(t)]>;
wherein:
mIINj is a normalized signal for a proposed placement for j-th volunteer.

18. The method of claim 1, wherein a standard lead I is calculated by:
Ii(t)=K1N(t)*[mINi(t)+mIINi(t)]−(ΔIi+ΔIIi);
wherein:
ΔIIi is a peak-to peak deviation of signal mII for i-th person outside from a learning group.

19. The method of claim 1, wherein the scaling coefficient for lead II is calculated by:
k2Nj(t)=<IINj/mIINj>, K2N(t)=<<k2Nj(t)>>;
wherein:
IINj is a normalized signal for the Standard lead II for j-th volunteer.

20. The method of claim 1, wherein the scaling coefficient for lead III is calculated by:
k3Nj(t)=<IIINj(t)/[mIIINj(t)−mIINj(t)]>, K3N(t)=<<k3Nj(t)>>;
IIIi(t)=K3N(t)*[mIIINi(t)−mIINi(t)]−(ΔIIIi−ΔIIi);
wherein:
IIINj is a normalized signal for a Standard lead III for j-th volunteer;
mINj is a normalized signal for a horizontal lead mIII for j-th volunteer; and
ΔIIIi is a peak-to peak deviation of signal mIII for i-th person outside from a learning group.
